(19) United States
(12) Patent Application Publication
(10) Pub. No.: US 2005/0025844 A1
(43) Pub. Date: Feb. 3, 2005

(54) Weight control compositions and methods

(76) Inventor: Matthias Boldt, Oxnard, CA (US)

Correspondence Address: JOSEPH E. CHOVANES, SUITE 329, 5 GREAT VALLEY PARKWAY, MALVERN, PA, 19355, US

(21) Appl. No.: 10/633,233

(22) Filed: Aug. 2, 2003

(51) Int. Cl. A61K 31/522; A61K 35/78

(52) U.S. Cl. 424/734; 514/263.32; 514/283

(57) ABSTRACT

The present invention provides compositions and methods that assist in providing weight control. Compositions comprise Caffeine, an adrenergic amine (e.g. synephrine, hordenine, octopamine, tyramine and N-methyltyramine,) forskolin, Guggulsterones, an alpha-2 receptor antagonist (e.g. Yohimbine) and a vinca alkaloid (e.g. vinpocetine.) Black pepper extract may be added as well in various alternative embodiments. Methods utilizing administration of nutrient compositions are disclosed as well.

### FIELD OF THE INVENTION

The present invention relates to weight control compositions and methods. More particularly, the present invention relates to weight control compositions and methods that provide aspects of fat loss as well as lean body mass maintenance.

### BACKGROUND OF THE INVENTION

Obesity is a problem in today's society. For example, it is estimated that the average American male has 22% to 25% fat as a percentage of body mass and the average American female has 33% to 35% fat as a percentage of body mass. Both averages are far beyond what is usually considered optimum, e.g., 15% to 19% for 20-29 year old males and 19% to 23% for 20-29 year old females.

Being obese or overweight either causes or promotes many serious health conditions. Therefore, it is desirable to attempt to assist people in losing weight through various types of treatments.

One possible treatment involves formulating weight loss products that appear to work similarly to some of the basic biochemical processes involved in fat metabolism. For example, artificial thermogenesis mimics the body's thermogenesis process. (Thermogenesis describes the process whereby food is converted to body heat through the metabolic process of caloric conversion. Too much undesired body fat may be caused through certain metabolic defects associated with the thermogenic process. Those metabolic defects may interfere with the conversion of food to body heat and so the food is stored as body fat.) Artificial thermogenesis does not convert food to body heat, as does thermogenesis. Rather, artificial thermogenesis converts body fat to body heat, by increasing the body's metabolic rate and causing it to burn fat.

Methods that may cause artificial thermogenesis are known in the art. For example, ephedra is an herb that grows wild in parts of the western United States. A ephedra derivative, ephedrine, is an alkaloid that stimulates the production of catecholamines such as norepinephrine. Norepinephrine or noradrenaline is presumed to start the artificial thermogenic process by stimulating metabolism in fat cells via the neurocrine axis that involves beta-adrenergic receptors, which in turn, results in lipolysis, or using fat cells to fuel an increase in the basal metabolic rate and body heat. Ephedrine may cause other difficulties, however, and so may be less than desirable for some individuals.

Moreover, simply causing artificial thermogenesis may be less than desirable as it may ignore other variables that are important in treating overweight and/or obese individuals. For example, the percentage of body composition that is not body fat is referred to as lean body mass. Lean body mass is comprised of muscle, vital organs, bone, connective and other non-fatty tissues in the body. An increase in lean body mass helps increase body metabolism. Thus, an increase in lean body mass will help in weight loss, and to maintain any weight reduction. Therefore, considering lean body mass is important for any weight loss strategy.

Artificial thermogenesis and other weight control means often do not take into account the importance of maintaining or increasing the lean body mass in the process of weight loss. In fact, regimens to decrease body fat often contribute to a decrease in lean body mass as well, and so slow down body metabolism causing attendant difficulties in maintaining an appropriate, healthy body weight. It would be desirable, therefore, to assist in providing control over the proportion of body fat to lean body mass.

Consequently, there is a need for compositions and methods that assist in providing control over the proportion of body fat to lean body mass.

### SUMMARY OF THE INVENTION

1) It is an object of the present invention to provide compositions and methods that assist in providing weight control. Weight control, as used herein, includes modification of the proportion of body fat to lean body mass. Weight control also includes decreasing body fat, as well as increasing or maintaining lean body mass. Embodiments may also be used to increase an individual's energy and/or stamina.

Compositions and methods are disclosed. A composition disclosed comprises: caffeine, an adrenergic amine, forskolin, guggulsterone, an alpha-2 receptor antagonist, and a vinca alkaloid. A method disclosed herein comprises administration of a compound comprising caffeine, an adrenergic amine, forskolin, guggulsterone, an alpha-2 receptor antagonist, and a vinca alkaloid.

Black pepper extract may also be used in certain alternative embodiments.

### DETAILED DESCRIPTION OF THE PREFERRED EMBODIMENTS

The present invention assists in providing control over the proportion of body fat to lean body mass. The preferred embodiments assist in causing body fat loss while building or maintaining lean body mass. Embodiments may also be used to increase an individual's energy and/or stamina.

The preferred embodiments comprise Caffeine Anhydrous (referred to herein as “caffeine”), an adrenergic amine (e.g. synephrine, hordenine, octopamine, tyramine and N-methyltyramine,) forskolin, Synthetic Guggulsterones Z and E (referred to herein as “Guggulsterones”), an alpha-2 receptor antagonist (e.g. Yohimbine) and a vinca alkaloid (e.g. vinpocetine.) Black pepper extract may be added as well in various alternative embodiments.

The preferred embodiments are administered in tablets, capsule or powders, in a therapeutically effective composition.

The following description may be helpful.

### Caffeine

The term “caffeine” as used herein is intended to encompass not only caffeine as the anhydrous powder, but any salt or derivative of caffeine or any compounded mixture thereof which is non-toxic and pharmaceutically acceptable. See, for example, The Merck Index, Merck & Co., Inc. Rahway, N.J. (9th Ed. 1976), pp. 207-208, for a description of caffeine salts, derivatives and mixtures that may prove useful in the compositions of the various embodiments. Nevertheless, caffeine as the anhydrous powder base is presently preferred and, where specific amounts of caffeine are set forth below, such amounts are given in mg of the anhydrous base.

The amount of caffeine in the compositions of the various preferred embodiments will typically be from about 10 mg to about 500 mg (preferably 200 to 400 mg) daily. It should be noted that this may be, in certain embodiments, administered in various doses throughout the day, (e.g. 2.5 mg to 125 mg 4 times.) It should also be noted that dosages may be varied as desired. For example, caffeine tolerance levels may alter the dose so that a relatively caffeine intolerant individual, for example, may take 10 mg, while a relatively caffeine tolerant individual may take 500 mg. Other predetermined variables as desired may also determine dosage.

### Synephrine

Synephrine is an adrenergic amine, and is utilized in the preferred embodiment of this invention, but synephrine may be substituted in various embodiments with one or more adrenergic amine selected from the following group consisting of: synephrine, hordenine, octopamine, tyramine and N-methyltyramine. These adrenergic amines, which help induce thermogenesis, are derived from citrus plants. The materials can be administered in their natural form or as extracts, in the form of capsules, tablets, powders, etc.

The amount of synephrine or other adrenergic amine in the compositions of the preferred embodiments will typically be from about 20 mg to 80 mg daily. It should be noted that this may be, in certain embodiments, administered in various doses throughout the day, (e.g. 5 to 20 mg 4 times.)

### Forskolin

Forskolin (also known as Forskohlin) is a chemically active ingredient derived from the herb Coleus forskohlii. Forskolin appears to increase the levels of cyclic AMP (cAMP) or exerts action similar to cAMP in the body, and so enhance the thermogenic response to food. An increase in the thermogenic response to food, in turn, improves absorption of nutrients and their preferential incorporation into lean body mass. Thus, the formation of lean body mass is promoted.

Forskolin can be administered in its natural form or as extracts, in the form of capsules, tablets, powders, etc. The amount of active forskolin content in the compositions of the preferred embodiments will vary due to the typically be from about 5 mg to 100 mg daily. It should be noted that this may be, in certain embodiments, administered in various doses throughout the day, (e.g. 1.25 to 25 mg 4 times.)

### Gugglesterones

Guggulsterones are standardized markers (also known as Guggulsterones E and Z) of Guggul gum resin from C. wightii. The compounds have also been isolated from the exudate of a plant known as Commiphora mukul (Hook, ex stocks) Engl. (syn. Balsamodendron mukul Hook) which is a small tree of the Burseraceae family. (In the ancient Sanskrit, the gum resin is called guggulu and is a product which is still used in Indian popular medicine for the treatment of obesity and some arthritic diseases.)

Guggulsterones can be administered in their natural form or as extracts, in the form of capsules, tablets, powders, etc. The amount of Guggulsterones in the compositions of the preferred embodiments will typically be from about 5 mg to 80 mg daily. It should be noted that this may be, in certain embodiments, administered in various doses throughout the day, (e.g. 1.25 to 20 mg 4 times.)

### Yohimbine

Yohimbine is synthesized from a Yohimbe tree (Pausinystalia yohimbe), a small evergreen tree native to Africa. Yohimbine is a potent naturally-occurring alpha-2 receptor antagonist and capable of increasing lipolysis in humans after oral dosing. It should be noted that other embodiments may use other alpha-2 receptor antagonists than, or in addition to, yohimbine.

Yohimbine can be administered in its natural form or as extracts, in the form of capsules, tablets, powders, etc. It should be noted that Yohimbine as used herein includes Yohimbee Bark as well. The amount of Yohimbine in the compositions of the preferred embodiments will typically be from about 5 mg to 25 mg daily. It should be noted that this may be, in certain embodiments, administered in various doses throughout the day, (e.g. 1.25 mg to 6.25 mg 4 times.)

### Vinpocetine

Vinpocetine (ethyl apovincaminate) is derived from Vincamine, an alkaloid extracted from the Periwinkle plant. Vinpocetine appears to increase carbohydrate metabolism, thus increasing levels of available energy. It should be noted that other embodiments may use other compounds which stimulate both glycolytic and oxidative reactions of glucose breakdown in the central nervous system (“CNS”), other than, or in addition to, such as Xanthinol Nicotinate and vincamine. Additionally, it should be noted that vinpocetine as used herein includes alkaloids of the Periwinkle plant as well. However, it should be understood that any other derivative of vincamine may be used in the compositions of various embodiments.

Vinpocetine can be administered in its natural form or as extracts, in the form of capsules, tablets, powders, etc. The amount of vinpocetine in the compositions of the preferred embodiments will typically be from about 5 mg to 25 mg daily. It should be noted that this may be, in certain embodiments, administered in various doses throughout the day, (e.g. 1.25 mg to 6.25 mg 4 times.)

It may also be desired, in alternative embodiments, to include Black Pepper Extract, in the form of Piperine or other compound. Black Pepper Extract is believed to act, directly or indirectly through activation of thermoreceptors, which results in increased thermogenesis, or metabolic heat energy production and release. Black Pepper Extract can be administered in its natural form or as extracts, in the form of capsules, tablets, powders, etc. The amount of Black Pepper Extract in the compositions of the preferred embodiments will typically be from about 5 to 25 mg daily. It should be noted that this may be, in certain embodiments, administered in various doses throughout the day, (e.g. 1.25 mg to 6.25 mg 4 times.)

### Formulation

A representative formula for an preferred embodiment comprises tablet, capsule and powders containing:

| | |
|---|---|
| Caffeine Anhydrous | 200 mg |
| Synephrine | 20 mg |
| Forskohlin | 20 mg |
| Guggulsterones | 20 mg |
| Yohimbine | 5 mg |
| Vinopocetine | 5 mg |

Of course this formula is suitable to variation in both the amount of a compound or the substitution of one of the above compounds in the composition with an analogous compound of similar function either as is described in this application or as known in the art.

Dosages may be varied as desired. For example, a single dose of the representative compound described above of 270 mg may be administered; some compounds may be administered in a single dose, with others in another dose administered substantially concomitantly; compounds may be administered separately, etc. Dosage forms may be as desired as well, e.g. tablets, capsules, etc. with appropriate additions as necessary or desired (e.g. excipients in a sufficient quantity of each to make a suitable tablet, etc.) Moreover, any compound used may have, in various dosage embodiments, various potencies and extracts ranging anywhere from 5% to 99% pure of active ingredient.

Dosages may be varied as desired. For example, a single dose of the representative compound described above may be administered; some compounds may be administered in a single dose, with others in another dose administered substantially concomitantly; compounds may be administered separately, etc. Dosage forms may be as desired as well, e.g. tablets, capsules, etc. with appropriate additions as necessary or desired (e.g. excipients in a sufficient quantity of each to make a suitable tablet, etc.)

### Administration

The dose can be given from 1 to about 6 times daily, preferably from 2 to about 4 times daily.

Compositions according to various embodiments may be formulated for administration by any suitable route such as the oral, rectal, nasal, topical (dermal) or parenteral administration route, and be in the form of tablets, capsules, suspensions, emulsions, solutions, injectables, suppositories, sprays, aerosols, sustained release compositions and/or devices, or others as desired.

Formulations for oral use include tablets which contain the active ingredient in admixture with non-toxic pharmaceutically acceptable excipients, as known in the art, for example, inert diluents, e.g. calcium carbonate, sodium chloride, lactose, calcium phosphate or sodium phosphate; granulating and disintegrating agents, e.g. potato starch or alginic acid; binding agents, e.g. starch, gelatin or acacia; lubricating agents, e.g., magnesium stearate, stearic acid or talc, etc.

Other pharmaceutically acceptable excipients can be colorants, flavouring agents, plasticizers, humectants etc. The tablets may be uncoated or they may be coated by known techniques, optionally to delay disintegration and absorption in the gastrointestinal tract and thereby provide a sustained action over a longer period. For example, a time delay material such as glyceryl monostearate or glyceryl distearate may be employed.

Formulations for oral use may also be presented as chewing tablets, or as hard gelatin capsules wherein the active ingredient is mixed with an inert solid diluent, for example, calcium carbonate, calcium phosphate or kaolin, or as soft gelatin capsules wherein the active ingredient is mixed with water or an oil medium, for example, peanut oil, liquid paraffin, or olive oil.

Powders, dispersible powders or granules suitable for preparation of an aqueous suspension by addition of water may also be used as convenient dosage forms. Formulation as a suspension provide the active ingredient in admixture with a dispersing or wetting agent, suspending agent and one or more preservatives. Suitable dispersing or wetting agents are, for example, naturally-occurring phosphatides, as e.g. lecithin, or condensation products of ethylene oxide with e.g. a fatty acid, a long chain aliphatic alcohol or a partial ester derived from fatty acids and a hexitol or a hexitol anhydrides, for example, polyoxyethylene stearate, polyoxyethylene sorbitol monooleate, polyoxyethylene sorbitan monooleate etc. Suitable suspending agents are, for example, sodium carboxymethylcellulose, methylcellulose, sodium alginate etc.

The pharmaceutical formulation may also be administered parenterally (intravenous, intramuscular, subcutaneous or the like) in dosage forms or formulations containing conventional, non-toxic pharmaceutically acceptable carriers and adjuvants, as known in the art.

Formulations for rectal application include suppositories (emulsion or suspension type), and rectal gelatin capsules (solutions or suspensions). Appropriate pharmaceutically acceptable suppository bases are used, such as cocoa butter, esterified fatty acids, glycerinated gelatin, and various water-soluble or dispersible bases like polyethylene glycols and polyoxyethylene sorbitan fatty acid esters. Various additives like e.g. enhancers or surfactants may be incorporated.

Formulations for nasal application include nasal sprays and aerosols for inhalation. In a typically nasal formulation, the active ingredients are dissolved or dispersed in a suitable vehicle. The pharmaceutically acceptable vehicles and excipients and optionally other pharmaceutically acceptable materials such as diluents, enhances, flavouring agents, preservatives etc. are all selected in accordance with conventional pharmaceutical practice in a manner understood by the persons skilled in the art of formulating pharmaceuticals.

Formulations for topical application for percutaneous absorption in dosage forms or formulations contain conventionally non-toxic pharmaceutically acceptable carriers and excipients including microspheres and liposomes as known in the art. The formulations include creams, ointments, lotions, liniments, gels, hydrogels, solutions, suspensions, pastes, plasters and other kinds of transdermal drug delivery systems. The pharmaceutically acceptable carriers or excipients may include emulsifying agents, antioxidants, buffering agents, preservatives, humectants, penetration enhancers, chelating agents, gelforming agents, ointment bases, perfumes and skin protective agents. Examples of emulsifying agents are naturally occurring gums, e.g. gum acacia or gum tragacanth, naturally occurring phosphatides, e.g. soybean lecithin and sorbitan monooleate derivatives. Examples of antioxidants are butylated hydroxy anisole (BHA), ascorbic acid and derivatives thereof, tocopherol and derivatives thereof and cysteine. Examples of preservatives are parabens and benzalkonium chloride. Examples of humectants are glycerin, propylene glycol, sorbitol and urea. Examples of penetration enhancers are propylene glycol, DMSO, triethanoiamine, N,N-dimethylacetamide, N,N-dimethylformamide, 2-pyrrolidone and derivatives thereof, tetrahydrofurfuryl alcohol and Azone. Examples of chelating agents are sodium EDTA, citric acid and phosporic acid. Examples of gel forming agents are Carbopol, cellulose derivatives, bentonit, alginates, gelatin and PVP. Examples of ointment bases are beeswax, paraffin, cetyl palmitate, vegetable oil, sorbitan esters of fatty acids (Span), polyethyleneglycols, and condensation products between sorbitan esters of fatty acids and ethylene oxide, e.g. polyoxyethylene sorbitan monooleate.

It should be understood that the ratios set forth herein are merely a preferred implementation and in no way limit the composition to the ratio described above, as people skilled in the art will recognize that variations in the ratio between substances will not significantly alter the properties of the present invention.

It should be further understood that this invention, and the aspects thereof, allow for substitution with a pharmaceutical and biochemical equivalent or complement, as long as that substitute functions in the same manner as the compound which it replaces in the composition.

2) A method for weight control in a mammal, comprising administration of a composition comprising: Caffeine, Adrenergic Amine, Forskolin, Guggulsterone, Alpha-2 Receptor Antagonist, and Vinca Alkaloid.

3) A method as in claim 1 further comprising Black Pepper Extract.

4) A method as in claim 1 wherein said Adrenergic Amine is selected from the group consisting of Synephrine, Hordenine, Octopamine, Tyramine And N-Methyltyramine.

5) A method as in claim 1 wherein said Alpha-2 Receptor Antagonist is Yohimbine.

6) A method as in claim 1 wherein said Vinca Alkaloid is Vinpocetine.

7) A method as in claim 1 wherein said administration comprises a therapeutically effective dosage.

8) A composition comprising Caffeine, Adrenergic Amine, Forskolin, Guggulsterone, Alpha-2 Receptor Antagonist, and Vinca Alkaloid.

9) A composition as in claim 7 further comprising Black Pepper Extract.

10) A composition as in claim 7 wherein said Adrenergic Amine is selected from the group consisting of Synephrine, Hordenine, Octopamine, Tyramine And N-Methyltyramine.

11) A composition as in claim 7 wherein said Alpha-2 Receptor Antagonist is Yohimbine.

12) A composition as in claim 7 wherein said Vinca Alkaloid is Vinpocetine.

13) A composition as in claim 7 wherein said administration comprises a therapeutically effective dosage.

14) The composition of claim 7 formulated for enteral administration comprising said unit dosage form admixed with flavors and sweeteners.

15) The composition of claim 7 formulated for enteral administration comprising said unit dosage form contained in blended powder or one or more capsules.

16) A composition according to claim 7 wherein said composition is in the form of a powder, tablet, capsule, pill, liquid, food additive, candy, confection or nutrition bar.

17) A composition according to claim 15 wherein said powder is admixed with a liquid.

18) The composition of claim 7 in a sustained release form.

19) An composition for enteral or parenteral administration comprising:
100 mg to about 500 mg by weight of Caffeine,
5 to 80 mg by weight of Adrenergic Amine,
5 mg to 100 mg by weight of Forskolin,
5 mg to 80 mg by weight of Guggulsterone,
5 mg to 50 mg by weight of Alpha-2 Receptor Antagonist, and
5 to 50 mg by weight of Vinca Alkaloid.

20) A method according to claim 1, wherein administering is performed on a daily basis.

21) A method according to claim 19, wherein said daily administration comprises at least two partial daily administrations of said composition.

22) A method according to claim 1, wherein administering is performed following an exercise period.

23) A method according to claim 1, wherein the composition comprises an amount of from about 125 mg to about 1310 g per day.

24) A method according to claim 1, wherein the composition comprises an amount of about 270 mg to about 810 mg per day.

25) A method as in claim 1 wherein said administration comprises a single daily administration.

26) A method as in claim 1 wherein said administration comprises partial daily administration, said partial daily administrations comprising at least one nutrient selected from the group comprising Caffeine, Adrenergic Amine, Forskolin, Guggulsterone, Alpha-2 Receptor Antagonist, and Vinca Alkaloid.

27) An composition consisting essentially of: 74.10% Caffeine, 7.41% Synephrine, 7.41% Forskolin, 7.41% Guggulsterones, 1.84% Yohimbine, and 1.84% Vinpocetine.

28) A composition according to claim 7 wherein said compound weights are adjusted according to predetermined factors.

29) A composition according to claim 27 wherein said predetermined factors include an individual's weight.

30) A composition according to claim 27 wherein said predetermined factors include an individual's exercise intensity.

31) A composition according to claim 27 wherein said predetermined factors include an individual's lean body mass.

32) A composition according to claim 27 wherein said predetermined factors include an individual's proportion of body fat to lean body mass.

33) A composition according to claim 27 wherein said predetermined factors include an individual's desire to increase energy.

34) A composition according to claim 27 wherein said predetermined factors include an individual's desire to increase stamina.
